(19) United States
(12) Patent Application Publication
(10) Pub. No.: US 2024/0408586 A1
(43) Pub. Date: Dec. 12, 2024

(54) METHOD FOR PRODUCING CATALYST COMPACT FOR USE IN PRODUCING UNSATURATED CARBOXYLIC ACID, AND METHOD FOR PRODUCING UNSATURATED CARBOXYLIC ACID AND UNSATURATED CARBOXYLIC ESTER USING SAME

(71) Applicant: MITSUBISHI CHEMICAL CORPORATION, Tokyo (JP)

(72) Inventors: Takuro WATANABE, Tokyo (JP); Wataru NINOMIYA, Tokyo (JP); Mitsunobu ITO, Tokyo (JP)

(73) Assignee: MITSUBISHI CHEMICAL CORPORATION, Tokyo (JP)

(21) Appl. No.: 18/812,991

(22) Filed: Aug. 22, 2024

Related U.S. Application Data
(63) Continuation of application No. PCT/JP2023/005171, filed on Feb. 15, 2023.

(30) Foreign Application Priority Data
Feb. 24, 2022 (JP) 2022-026283

(51) Int. Cl. B01J 27/199; B01J 37/02; C07C 51/16; C07C 67/04

(52) U.S. Cl. CPC B01J 27/199; B01J 37/0213; C07C 51/16; C07C 67/04

(57) ABSTRACT

An object is to provide a method for producing a catalyst molded article which is high in mechanical strength and which enables unsaturated aldehyde and/or unsaturated carboxylic acid to be produced at high yield(s). The object is achieved by a method for producing a catalyst molded article, the method including (1) a step of adding an ammonium radical to a slurry (liquid A) including molybdenum and phosphorus, at a rate satisfying the following expression (I), to prepare a slurry (liquid B), 0.1≤v/M≤3 (I), in which v represents the rate of addition of the ammonium radical [mol/hour], and M represents the mass of the liquid A [kg], (2) a step of adjusting the pH of the liquid B, to prepare a slurry (liquid C) having a pH lower than that of the liquid B by 0.2 or more and having a pH of 4 or less, (3) a step of drying the liquid C, to obtain a catalyst powder, (4) a step of mixing the catalyst powder and a liquid, to produce a catalyst powder mixture, and (5) a step of molding the catalyst powder mixture, to produce a catalyst molded article, in which the penetration rate coefficient of the liquid to the catalyst powder in step (4) is 0.07 g²/s or more.

### CROSS-REFERENCE TO RELATED APPLICATION

This is a continuation of International Application PCT/JP2023/005171, filed on Feb. 15, 2023, and designated the U.S., and claims priority from Japanese Patent Application 2022-026283 which was filed on Feb. 24, 2022, the entire contents of which are incorporated herein by reference.

### TECHNICAL FIELD

The present invention relates to a method for producing a catalyst molded article for use in the production of unsaturated carboxylic acid, and method for producing unsaturated carboxylic acid and unsaturated carboxylic acid ester.

### BACKGROUND ART

In production processes of unsaturated carboxylic acid, spherical catalyst molded articles each having a diameter of about 2 to 20 mm, or columnar or cylinder catalyst molded articles each having a diameter of about 2 to 10 mm and a length of about 2 to 20 mm are generally used, and oxidation reaction is performed with reactors filled with such catalyst molded articles.

For the production of catalyst molded articles, for example, PTL 1 proposes a method for producing a catalyst for methacrylic acid production, including molybdenum and phosphorus as catalyst components, the method including a step of drying an aqueous mixed liquid including raw material compounds as the catalyst components, to produce a dried product having an apparent density (X) of 1.00 to 1.80 kg/L, and a step of molding the dried product or a mixture including the dried product, to produce a catalyst molded article having a molded product density (Y) of 1.60 to 2.40 kg/L and a ratio (X/Y) of the apparent density (X) and the molded product density (Y), of 0.50 to 0.80.

### RELATED ART DOCUMENTS

### Patent Documents

PTL 1: WO 2012/141076

### SUMMARY OF THE INVENTION

However, there is a demand for a catalyst molded article enabling an objective product to be produced at a high yield over a longer period, from an industrial viewpoint. In order to satisfy the demand, a catalyst molded article is needed which not only provides an excellent yield of an objective product, but also is high in mechanical strength. When a catalyst molded article is high in mechanical strength, such a catalyst molded article is reduced in pulverization thereof in long-term continuous running, and thus is inhibited from being increased in differential pressure in a reactor and can provide a yield kept over a long period.

An object of the present invention is to provide a catalyst molded article which is high in mechanical strength and which enables unsaturated carboxylic acid to be produced at a high yield. Another object of the present invention is to provide methods for producing unsaturated carboxylic acid and unsaturated carboxylic acid ester with the catalyst molded article.

The present inventors have made intensive studies in view of the above problems. As a result, the inventors have found that the above problems can be solved by producing a catalyst molded article with a dried catalyst powder having a specified penetration rate coefficient, and thus have completed the present invention.

Specifically, the present invention includes the following.
[1]: A method for producing a catalyst molded article for use in the production of unsaturated carboxylic acid by oxidation reaction of unsaturated aldehyde, the method including
(1) a step of adding an ammonium radical to a solution or slurry (liquid A) including molybdenum and phosphorus, at a rate satisfying the following expression (I), to prepare a solution or slurry (liquid B),

$$0.1 \leq v / M \leq 3 \qquad ( I )$$

wherein v represents the rate of addition of the ammonium radical [mol/hour], and M represents the mass of the liquid A [kg],
(2) a step of adjusting the pH of the liquid B, to prepare a solution or slurry (liquid C) having a pH lower than that of the liquid B by 0.2 or more and having a pH of 4 or less,
(3) a step of drying the liquid C, to obtain a catalyst powder,
(4) a step of mixing the catalyst powder and a liquid, to produce a catalyst powder mixture, and
(5) a step of molding the catalyst powder mixture, to produce a catalyst molded article, wherein the penetration rate coefficient of the liquid to the catalyst powder in step (4) is 0.07 g²/s or more.
[2]: The method for producing a catalyst molded article according to [1], wherein the penetration rate coefficient of the liquid to the catalyst powder in step (4) is 0.073 to 0.15 g²/s.
[3]: The method for producing a catalyst molded article according to [1] or [2], wherein the liquid contains one selected from the group consisting of water and an alcohol having 1 to 4 carbon atoms, in step (4).
[4]: The method for producing a catalyst molded article according to any of [1] to [3], wherein the liquid contains 50% by mass or more of one selected from the group consisting of water and an alcohol having 1 to 4 carbon atoms, in step (4).
[5]: The method for producing a catalyst molded article according to any of [1] to [4], wherein the catalyst powder mixture is extrusion-molded to produce the catalyst molded article in step (5).
[6]: The method for producing a catalyst molded article according to any of [1] to [5], wherein the pH of the liquid A is 3 or less in step (1).
[7]: The method for producing a catalyst molded article according to any of [1] to [6], wherein the v/M is 0.2 to 2 in step (1).
[8]: The method for producing a catalyst molded article according to any of [1] to [7], wherein the liquid B is stirred for 5 minutes or more and then the pH is adjusted in step (2).
[9]: The method for producing a catalyst molded article according to any of [1] to [8], wherein the liquid C having a pH lower than that of the liquid B by 0.5 or more is prepared in step (2).
[10]: The method for producing a catalyst molded article according to any of [1] to [9], wherein the liquid C having a pH of 3.5 or less is prepared in step (2).
[11]: The method for producing a catalyst molded article according to any of [1] to [10], wherein the catalyst powder has a composition represented by the following formula (II):

PₐMo_{b}V_{c}Cu_{d}X1ₑY1_{f}Z1_{g}(NH₄)ₕOᵢ (II)
wherein P, Mo, V, Cu, NH₄ and O respectively represent phosphorus, molybdenum, vanadium, copper, ammonium radical and oxygen, X1 represents at least one element selected from the group consisting of silicon, germanium, arsenicum, antimony, and bismuth, Y1 represents at least one element selected from the group consisting of niobium, tungsten, iron, zinc, chromium, cobalt and manganese, Z1 represents at least one element selected from the group consisting of lithium, sodium, potassium, rubidium and cesium, a to i each represent the molar ratio of each component, b=12, a=0.5 to 3, c=0.01 to 3, d=0.01 to 2, e=0 to 3, f=0 to 3, g=0.01 to 3, and h=0.01 to 30 are satisfied, and i is the molar ratio of oxygen necessary for satisfying the valence of such each component.
[12]: A method for producing unsaturated carboxylic acid, including producing unsaturated carboxylic acid with a catalyst molded article produced by the method according to any [1] to [11], by oxidation reaction of unsaturated aldehyde.
[13]: A method for producing unsaturated carboxylic acid ester, including esterifying unsaturated carboxylic acid produced by the method according to [12].

### Effects of the Invention

According to the present invention, there can be provided a catalyst molded article which is high in mechanical strength and which enables unsaturated carboxylic acid to be produced at a high yield.

### MODE FOR CARRYING OUT THE INVENTION

Hereinafter, embodiments of the present invention are described in detail, but the description of constituent requirements described below is illustrative for carrying out aspects of the present invention, and the present invention is not identified by these contents. Expressions representing numerical value ranges, such as “XX or more and YY or less” and “XX to YY”, each means such a numerical value range including the lower limit and the upper limit as end points, unless particularly noted. In a case where these numerical value ranges are described stepwise, the upper limits and the lower limits of these respective numerical value ranges can be arbitrarily combined. While the invention has been described in detail and with reference to specific embodiments thereof, it will be apparent to one skilled in the art that various changes and modifications can be made therein without departing from the spirit and scope thereof.

### [Catalyst Molded Article]

A catalyst molded article obtained by a method for producing a catalyst molded article according to the present embodiment is used in the production of unsaturated carboxylic acid by oxidation reaction of unsaturated aldehyde.

The catalyst molded article preferably includes a catalyst powder having a composition represented by the following formula (II), from the viewpoint of the yield of an objective product. Thus, unsaturated carboxylic acid can be produced at a high yield. The catalyst powder may include a small amount of any element not described in the following formula (II).

PₐMo_{b}V_{c}Cu_{d}X1ₑY1_{f}Z1_{g}(NH₄)ₕOᵢ (II)

In the formula (II), P, Mo, V, Cu, NH₄ and O respectively represent phosphorus, molybdenum, vanadium, copper, ammonium radical, and oxygen. X1 represents at least one element selected from the group consisting of silicon, germanium, arsenicum, antimony, and bismuth. Y1 represents at least one element selected from the group consisting of niobium, tungsten, iron, zinc, chromium, cobalt, and manganese. Z1 represents at least one element selected from the group consisting of lithium, sodium, potassium, rubidium and cesium. a to i each represent the molar ratio of each component, b=12, a=0.5 to 3, c=0.01 to 3, d=0.01 to 2, e=0 to 3, f=0 to 3, g=0.01 to 3, and h=0.01 to 30 are satisfied, and i is the molar ratio of oxygen necessary for satisfying the valence of such each component.

In the formula (II), from the viewpoint of an enhancement in yield of unsaturated carboxylic acid, the lower limit of a is preferably 0.6 or more, more preferably 0.7 or more. The upper limit of a is preferably 2.5 or less, more preferably 2 or less. The lower limit of c is preferably 0.1 or more, preferably 0.15 or more, further preferably 0.2 or more. The upper limit of c is preferably 2.5 or less, more preferably 2 or less, further preferably 1.5 or less. The lower limit of d is preferably 0.03 or more, more preferably 0.05 or more. The upper limit of d is preferably 2.5 or less, more preferably 2 or less. The lower limit of e is preferably 0.01 or more, more preferably 0.1 or more. The upper limit of e is preferably 2.5 or less, more preferably 2 or less. The upper limit of f is preferably 2 or less, more preferably 1.5 or less, further preferably 1 or less. The lower limit of g is preferably 0.1 or more, more preferably 0.3 or more. The upper limit of g is preferably 2.8 or less, more preferably 2.5 or less. The upper limit of h is preferably 15 or less, more preferably 10 or less.

The molar ratio of each component is a value determined by analyzing each component of the catalyst dissolved in ammonia water, with ICP emission spectroscopy. The molar ratio of the ammonium radical is a value determined by analyzing the catalyst according to the Kjeldahl method. The ammonium radical in the present invention is a collective term of ammonia (NH₃) capable of being converted into an ammonium ion (NH₄⁺), and ammonium contained in an ammonium-containing compound such as an ammonium salt.

### [Method for Producing Catalyst Molded Article]

A method for producing a catalyst molded article according to the present embodiment is a method for producing a catalyst molded article for use in the production of unsaturated carboxylic acid by oxidation reaction of unsaturated aldehyde, the method including the following steps (1) to (5).

(1) A step of adding an ammonium radical to a solution or slurry (liquid A) including molybdenum and phosphorus, at a rate satisfying the following expression (I), to prepare a solution or slurry (liquid B),

$$0.1 \leq v / M \leq 3 \qquad ( I )$$

wherein v represents the rate of addition of the ammonium radical [mol/hour], and M represents the mass of the liquid A [kg].

(2) A step of adjusting the pH of the liquid B, to prepare a solution or slurry (liquid C) having a pH lower than that of the liquid B by 0.2 or more and having a pH of 4 or less.

(3) A step of drying the liquid C, to obtain a catalyst powder.

(4) A step of mixing the catalyst powder and a liquid, to produce a catalyst powder mixture.

(5) A step of molding the catalyst powder mixture, to produce a catalyst molded article.

In the method for producing a catalyst molded article according to the present embodiment, the penetration rate coefficient of the liquid to the catalyst powder in step (4) is 0.07 g²/s or more.

A catalyst molded article produced by this method is high in mechanical strength and enables unsaturated carboxylic acid to be produced at a high yield.

Hereinafter, each step is described in detail.

### (Step (1))

In step (1), an ammonium radical is added to a solution or slurry (liquid A) including molybdenum and phosphorus, at a rate satisfying the expression (I), to prepare a solution or slurry (liquid B).

### <Liquid A>

The liquid A can be prepared by dissolving or suspending a raw material compound including molybdenum and phosphorus in a solvent. The amount of the raw material compound used is preferably adjusted so that a catalyst powder obtained in step (3) described below has the composition represented by the formula (II).

The type of the raw material compound is not particularly limited, and oxide, sulfate, nitrate, carbonate, hydroxide, an organic acid salt such as acetate, an ammonium salt, halide, oxyacid, an oxyacid salt, an alkali metal salt, or the like of each constituent element can be used singly or in combination of two or more kinds thereof. Examples of the raw material compound of molybdenum include molybdenum oxides such as molybdenum trioxide, ammonium molybdates such as ammonium paramolybdate and ammonium dimolybdate, molybdic acid, and molybdenum chloride. Examples of the raw material compound of phosphorus include phosphoric acid, phosphorus pentoxide, and phosphates such as ammonium phosphate. Examples of the raw material compound of vanadium include ammonium vanadate, ammonium metavanadate, vanadium pentoxide, vanadium chloride, and vanadyl oxalate. Examples of the raw material compound of copper include copper sulfate, copper nitrate, copper oxide, copper carbonate, copper acetate, and copper chloride.

Examples of the solvent include water, ethyl alcohol, and acetone. These may be used singly or in combination of two or more kinds thereof. In particular, water is preferably used.

In a case where a catalyst powder having the composition represented by the formula (II) is produced, the liquid A is preferably prepared by dissolving or suspending the raw material compound including molybdenum and phosphorus in the solvent, to prepare a solution or slurry (liquid A1), and mixing the liquid A1 obtained and a raw material compound of the element Z1 in the formula (II).

The pH of the liquid A1 is preferably 0.3 to 5.5, the lower limit is more preferably 0.5 or more and the upper limit is more preferably 4.5 or less. The pH can be adjusted by appropriately selecting the type and amount of a raw material compound used and, if necessary, adding nitric acid, oxalic acid, or the like. The pH can be measured with a pH meter. The pH meter here used can be, for example, D-21 (product name, manufactured by Horiba Ltd.).

The liquid A1 is preferably heated to 80 to 130° C. and thus prepared. When the heating temperature of the liquid A1 is 80° C. or more, the dissolution rate of a catalyst raw material can be sufficiently increased. When the heating temperature of the liquid A1 is 130° C. or less, evaporation of the solvent can be suppressed. The lower limit of the heating temperature of the liquid A1 is more preferably 90° C. or more.

The pH of the liquid A obtained by mixing the liquid A1 and the raw material compound of the element Z1 is preferably 3 or less, the lower limit is more preferably 0.5 or more and the upper limit is more preferably 2.5 or less.

The temperature of the liquid A1 in mixing of the liquid A1 and the raw material compound of the element Z1 is preferably 30 to 99° C. Thus, when an objective product is produced with the catalyst obtained, local generation of heat in the catalyst can be suppressed. The lower limit of the temperature of the liquid A1 is more preferably 40° C. or more and the upper limit thereof is more preferably 95° C. or less.

### <Liquid B>

The liquid B is prepared by adding an ammonium radical to the liquid A at a rate satisfying the following expression (I).

$$0.1 \leq v / M \leq 3 \qquad ( I )$$

In the formula (I), v represents the rate of addition of the ammonium radical [mol/hour], and M represents the mass of the liquid A [kg]. In a case where the ammonium radical is added to the liquid A through a plurality of addition ports, v is a value based on the total number of moles of the ammonium radical added through the plurality of addition ports.

The liquid B thus obtained can be subjected to pH adjustment in step (2) described below, thereby easily obtaining a catalyst powder having a desired penetration rate coefficient in step (3) described below. In the expression (I), the v/M is preferably 0.2 to 2, and the lower limit is more preferably 0.3 or more and the upper limit is more preferably 1 or less.

Examples of the raw material compound of the ammonium radical include ammonium bicarbonate, ammonium carbonate, ammonium nitrate, and ammonia water.

The temperature of the liquid A in mixing of the ammonium radical is preferably 30 to 99° C. Thus, when an objective product is produced with the catalyst obtained, local generation of heat in the catalyst can be suppressed. The lower limit of the temperature of the liquid A is more preferably 40° C. or more and the upper limit thereof is more preferably 95° C. or less.

The pH of the liquid B obtained is preferably 0.3 to 5.5, and the lower limit is more preferably 1 or more and the upper limit is more preferably 5 or less.

### (Step (2))

In step (2), the pH of the liquid B obtained in step (1) is adjusted, to prepare a solution or slurry (liquid C) having a pH lower than that of the liquid B by 0.2 or more and having a pH of 4 or less. Thus, a catalyst powder having a desired penetration rate coefficient can be easily obtained in step (3) described below.

The pH is adjusted after stirring of the liquid B for preferably 5 minutes or more, more preferably 10 minutes or more, from the viewpoint of stabilization of the penetration rate coefficient of such the catalyst powder. The upper limit of the time for stirring the liquid B is not particularly restricted, and can be, for example, 60 minutes or less. The stirring can be performed with a rotor stirrer or a magnetic stirrer.

The pH of the liquid B can be adjusted by, for example, addition of an acid. Examples of the acid added include nitric acid and oxalic acid.

The pH of the liquid C obtained is preferably lower than that of the liquid B by 0.5 or more, more preferably by 1 or more, further preferably by 1.5 or more. The pH of the liquid C is preferably 3.5 or less, more preferably 3 or less, further preferably 2.5 or less.

### (Step (3))

In step (3), the liquid C obtained in step (2) is dried, to obtain a catalyst powder. The drying method is not particularly limited, and, for example, a drying method with a spray dryer, a drying method with a slurry dryer, a drying method with a drum dryer, or an evaporation drying method can be applied. In particular, a drying method with a spray dryer is preferred because a particle is obtained at the same time as drying and the shape of the particle obtained is a neat spherical shape.

Drying conditions are varied depending on the drying method, and in the case of use of a spray dryer, the dryer inlet temperature is preferably 200 to 400° C., and the lower limit is more preferably 220° C. or more and the upper limit is more preferably 370° C. or less. The contact system of droplets sprayed and hot air may be any of co-current flow, countercurrent flow, and co-current/countercurrent flow (mixed flow), and even any case can provide suitable drying.

The average particle size of the catalyst powder obtained is preferably 1 to 250 μm. When the average particle size is 1 μm or more, a pore size necessary for oxidation reaction can be ensured and an objective product is obtained at a high yield. When the average particle size is 250 μm or less, the number of contact points between catalyst powder particles per unit volume is kept and a reduction in mechanical strength of the catalyst molded article is suppressed. The lower limit of the average particle size of the catalyst powder is more preferably 5 μm or more and the upper limit thereof is more preferably 150 μm or less. The average particle size means the volume average particle size and is a value measured with a laser type particle size distribution measurement apparatus.

The catalyst powder obtained may be, if necessary, heat-treated at 200 to 500° C. Such heat treatment is usually performed under flow of oxygen, air or nitrogen. The heat treatment time is appropriately set depending on the intended catalyst.

### (Step (4))

In step (4), the catalyst powder obtained in step (3) and a liquid are mixed, to produce a catalyst powder mixture.

### <Liquid>

The liquid in the present invention means a compound which is liquid under conditions of ordinary temperature (5 to 40° C.) and ordinary pressure (atmospheric pressure (0.1 MPa)).

The liquid used in step (4) is not particularly limited as long as it has the function of wetting the catalyst powder. In particular, a liquid including at least one selected from the group consisting of water and an alcohol having 1 to 4 carbon atoms is preferably used because such use does not cause a collapse of particles of the catalyst powder, and pores that are effective for oxidation reaction are easily formed. The liquid more preferably includes 50% by mass or more, further preferably 80% by mass or more, particularly preferably 90% by mass or more of one selected from the group consisting of water and an alcohol having 1 to 4 carbon atoms. The alcohol having 1 to 4 carbon atoms, here used, is preferably ethyl alcohol or propyl alcohol.

The amount of the liquid used is appropriately selected depending on the type and size of the catalyst powder, the type of the liquid, and the like, and is preferably 10 to 80 parts by mass based on 100 parts by mass of the catalyst powder.

### <Penetration Rate Coefficient of Catalyst Powder>

The penetration rate coefficient of the catalyst powder to the liquid used in step (4) is 0.07 g²/s or more. Thus, a mixture in the state where the liquid is appropriately absorbed by the catalyst powder is obtained regardless of the type of the liquid. It is considered that the mixture is molded in step (5) described below, resulting in an enhancement in binding ability between catalyst powder particles and obtaining a catalyst molded article high in mechanical strength. Furthermore, it is considered that, since the collapse of catalyst powder particles in molding can be suppressed, a catalyst molded article having many pores effective for production of an objective product is obtained, and an objective product can be produced at a high yield. The penetration rate coefficient is the rate coefficient of the liquid penetrating into a powder layer. The lower limit of the penetration rate coefficient of the catalyst powder is preferably 0.073 g²/s or more, more preferably 0.076 g²/s or more. The upper limit of the penetration rate coefficient is preferably 0.15 g²/s or less.

The penetration rate coefficient can be determined from the slope of the fitted curve under the assumption that the penetration time and the square of the weight of a penetrating liquid are in a linear relation. The slope of the fitted curve is determined from the slope obtained by linear approximation of the curve in a region corresponding to two-thirds or less of the saturated amount of the weight of penetration. The penetration rate coefficient can be measured with, for example, Peneto Analyzer PNT-N manufactured by Hosokawa Micron Corporation or Processor Tensiometer K100 manufactured by KRUSS. Here, a measurement cell having a diameter of 36 mm is used in measurement of the penetration rate coefficient. The penetration rate coefficient of the catalyst powder is measured at room temperature after compression of the catalyst powder in the measurement cell by a 200-g weight and, at the same time, tapping 50 times of the catalyst powder at a stroke of 18 mm.

The penetration rate coefficient of the catalyst powder to the liquid can be controlled by, for example, a method including adjusting the amount of the raw material compound used, to change the composition of the catalyst powder or change the surface state of the catalyst powder, in step (1), and is preferably controlled by such a method including changing the surface state of the catalyst powder.

In a case where the composition of the catalyst powder is modified, for example, in a case where the catalyst powder, which has the composition represented by the formula (II), is used, the penetration rate coefficient tends to be increased by a reduction in cl as the molar ratio of vanadium.

The surface state of the catalyst powder can be controlled by, for example, modifying the v/M in addition of the ammonium radical in step (1), or modifying the pH of the liquid C in step (2). When the v/M in step (1) is increased, the penetration rate coefficient of the catalyst powder obtained tends to be decreased. When the pH of the liquid C is adjusted to a lower pH in step (2), the penetration rate coefficient of the catalyst powder obtained tends to be increased.

The surface state of the catalyst powder can also be controlled by modifying the drying temperature of the liquid C in step (2). When the drying temperature is higher, the penetration rate coefficient of the catalyst powder obtained tends to be increased.

### <Mixing of Catalyst Powder and Liquid>

The mixing in step (4) means an operation for admixing the catalyst powder and the liquid. The mixing method is not particularly limited, and examples include a method including kneading the catalyst powder and the liquid with a kneader. A method may also be used which includes spraying the liquid to the catalyst powder with a pan-type granulator.

Polyvinyl alcohol, an α-glucan derivative, a β-glucan derivative, stearic acid, ammonium nitrate, graphite, water, an alcohol, or the like can be, if necessary, added as a molding aid during the mixing.

### (Step (5))

In step (5), the catalyst powder mixture obtained in step (4) is molded, to produce a catalyst molded article. The molding method is not particularly limited, and examples include known methods such as extrusion molding, tablet molding, support molding, and rolling granulation. In particular, extrusion molding is preferred because pores effective for production of an objective product are formed in the catalyst molded article. For example, an auger-type extrusion molding machine or a plunger-type extrusion molding machine can be used in extrusion molding, and a plunger-type extrusion molding machine is preferably used. Thus, the number of pores formed in the catalyst molded article is increased.

In a case where the catalyst powder mixture is extrusion-molded to produce the catalyst molded article, the extrusion pressure is preferably 0.1 to 30 MPa (G). Herein, (G) means the gauge pressure. When the extrusion pressure is 0.1 MPa (G) or more, a catalyst molded article having a certain shape can be stably produced. When the extrusion pressure is 30 MPa or less, pores formed in the catalyst molded article are inhibited from being decreased. The lower limit of extrusion pressure is more preferably 0.5 MPa (G) or more, further preferably 1 MPa (G) or more, particularly preferably 2 MPa (G) or more. The upper limit of the extrusion pressure is more preferably 20 MPa (G) or less, further preferably 15 MPa (G) or less, particularly preferably 10 MPa (G) or less.

The catalyst molded article obtained may be, if necessary, dried at 50 to 120° C., to remove a liquid.

As described above, a catalyst molded article can be produced which is high in mechanical strength and which enables unsaturated carboxylic acid to be produced at a high yield.

### [Method for Producing Unsaturated Carboxylic Acid]

A method for producing unsaturated carboxylic acid according to the present embodiment includes using a catalyst molded article produced by the method for producing a catalyst molded article according to the present embodiment, to perform oxidation reaction of unsaturated aldehyde. Thus, unsaturated carboxylic acid can be produced at a high yield.

Examples of the unsaturated aldehyde include (meth)acrolein, crotonaldehyde (β-methyl acrolein), and cinnamaldehyde (β-phenyl acrolein).

The unsaturated carboxylic acid produced is unsaturated carboxylic acid in which an aldehyde group of the unsaturated aldehyde is converted into a carboxyl group. For example, in a case where the unsaturated aldehyde is (meth)acrolein, (meth)acrylic acid is obtained. The unsaturated aldehyde and/or the unsaturated carboxylic acid are/is respectively preferably (meth)acrolein and (meth)acrylic acid, more preferably methacrolein and methacrylic acid from the viewpoint of the yield of an objective product. Herein, the “(meth)acrolein” represents acrolein and methacrolein, and the “(meth)acrylic acid” represents acrylic acid and methacrylic acid.

In the method for producing unsaturated carboxylic acid according to the present embodiment, a catalyst molded article produced by the method for producing a catalyst molded article according to the present embodiment is preferably fired and then used from the viewpoint of the yield of an objective product. The firing temperature is usually 200 to 600° C., and the lower limit is preferably 300° C. or more and the upper limit is preferably 500° C. or less. Firing conditions are not particularly limited, and the firing is usually performed under flow of oxygen, air or nitrogen. The firing time is appropriately set depending on the intended catalyst, and is preferably 0.5 to 40 hours, and the lower limit is more preferably 1 hour or more and the upper limit is more preferably 40 hours or less.

Hereinafter, there is described, as a representative example, a method for producing methacrylic acid by oxidation reaction of methacrolein with a catalyst molded article produced by the method for a catalyst molded article according to the present embodiment.

In the method, methacrylic acid is produced by contacting a raw material gas including methacrolein and oxygen, and the catalyst molded article in a reactor. The reactor here used can be a fixed bed-type reactor. The oxidation reaction can be performed by filling the reactor with the catalyst molded article and feeding the raw material gas to the reactor. The catalyst molded article layer may be a single layer, or a plurality of such catalyst molded articles different in activity may be respectively divided to a plurality of layers and subjected to filling. The catalyst molded article may be diluted with an inert carrier and subjected to filling in order to control activity.

The concentration of methacrolein in the raw material gas is not particularly limited and is preferably 1 to 20% by volume, the lower limit is more preferably 3% by volume or more, and the upper limit is more preferably 10% by volume or less. Here, methacrolein may include a small amount of impurities having no substantial influence on the present reaction of lower saturated aldehyde or the like.

The concentration of oxygen in the raw material gas based on 1 mol of methacrolein is preferably 0.4 to 4 mol, the lower limit is more preferably 0.5 mol or more, and the upper limit is more preferably 3 mol or less. The oxygen source is preferably air from the viewpoint of economic performance. If necessary, an oxygen-rich gas may be used in which pure oxygen is added to the air.

The raw material gas may be one in which methacrolein and oxygen are diluted with an inert gas such as nitrogen or carbon dioxide. Water vapor may also be further added to the raw material gas. Methacrylic acid can be obtained at a high yield by reaction performed in the presence of water vapor. The concentration of water vapor in the raw material gas is preferably 0.1 to 50% by volume, the lower limit is more preferably 1% by volume, and the upper limit is more preferably 40% by volume.

The contact time between the raw material gas and the catalyst molded article is preferably 1.5 to 15 seconds, and the lower limit is more preferably 2 seconds or more and the upper limit is more preferably 10 seconds or less. The reaction pressure is preferably 0.1 to 1 MPa (G). The reaction temperature is preferably 200 to 450° C., and the lower limit is more preferably 250° C. or more and the upper limit is more preferably 400° C. or less.

Methacrylic acid can be obtained at a high yield by production as described above.

### [Method for Producing Unsaturated Carboxylic Acid Ester]

A method for producing unsaturated carboxylic acid ester according to the present embodiment includes esterifying unsaturated carboxylic acid produced by the method for producing unsaturated carboxylic acid according to the present embodiment. In other words, the method for producing unsaturated carboxylic acid ester according to the present embodiment includes a step of producing unsaturated carboxylic acid by the method for producing unsaturated carboxylic acid according to the present embodiment and a step of esterifying the unsaturated carboxylic acid. According to such a method, unsaturated carboxylic acid ester can be obtained with unsaturated carboxylic acid obtained by oxidation reaction of alkene, alcohol or ether, or oxidation reaction of unsaturated aldehyde.

The alcohol to be allowed to react with the unsaturated carboxylic acid is not particularly limited, and examples include methanol, ethanol, isopropanol, n-butanol, and isobutanol. Examples of the unsaturated carboxylic acid ester obtained include methyl (meth)acrylate, ethyl (meth)acrylate, propyl (meth)acrylate, and butyl (meth)acrylate. Such reaction can be performed in the presence of an acidic catalyst such as a sulfonic acid-type cation-exchange resin. The reaction temperature is preferably 50 to 200° C.

### EXAMPLES

Hereinafter, the present invention is specifically described with reference to Examples and Comparative Examples, but the present invention is not limited to these Examples. Herein, “part(s)” represents “part(s) by mass”.

### (Composition of Catalyst Powder)

The molar ratio of each element in the catalyst powder was determined by analyzing each component of the catalyst powder dissolved in ammonia water, with ICP emission spectroscopy. The molar ratio of the ammonium radical was determined by analyzing the catalyst powder according to the Kjeldahl method.

### (Penetration Rate Coefficient of Catalyst Powder)

The penetration rate coefficient of the catalyst powder was measured with Peneto Analyzer PNT-N manufactured by Hosokawa Micron Corporation, and a measurement cell having a diameter of 36 mm. The penetration rate coefficient of the catalyst powder was measured at room temperature after compression of the catalyst powder in the measurement cell by a 200-g weight and, at the same time, tapping 50 times of the catalyst powder at a stroke of 18 mm.

### (Mechanical Strength of Catalyst Molded Article)

The falling pulverization ratio of the catalyst molded article was used as an index of the mechanical strength of the catalyst molded article. It is indicated that, as the falling pulverization ratio is smaller, the mechanical strength is higher, and as the falling pulverization ratio is larger, the mechanical strength is lower. The falling pulverization ratio of the catalyst molded article was measured by the following method.

One hundred g of the catalyst molded article was allowed to fall through an upper opening of a stainless cylinder which was disposed so that its longitudinal direction was perpendicular and which had a lower opening closed with a stainless plate and had an inner diameter of 27.5 mm and a length of 6 m, and thus the cylinder was filled with the catalyst molded article. The falling pulverization ratio was calculated by the following expression where the mass of the catalyst molded article recovered with the lower opening being opened, which did not pass through a sieve having an aperture of 1 mm, was defined as Mg. The falling pulverization ratio in Examples was the average value of the falling pulverization ratio with respect to each of such catalyst molded articles produced 10 times under the same conditions.

Falling pulverization ratio (%)={(100−M)/100}×100

### (Reaction Evaluation)

Reaction evaluation of the catalyst molded article was performed with, as an example, production of methacrylic acid by oxidation reaction of methacrolein. The raw material gas and the product in the reaction evaluation were analyzed with gas chromatography (apparatus: GC-2014 manufactured by Shimadzu Corporation, column: DB-FFAP manufactured by J&W, 30 m×0.32 mm, film thickness 1.0 μm). The yield of methacrylic acid produced was calculated from the results of gas chromatography, according to the following expression.

Yield of methacrylic acid (%)=(N2/N1)×100

Here, N1 represents the number of moles of methacrolein fed, and N2 represents the number of moles of methacrylic acid produced.

### Example 1

To 4000 parts of pure water were added 1000 parts of molybdenum trioxide, 54 parts of ammonium metavanadate, 93.5 parts of an aqueous 85% by mass phosphoric acid solution, and 7 parts of copper nitrate. The resulting slurry was heated to 95° C. under stirring and stirred for 3 hours with the liquid temperature being kept at 95° C., and thus a liquid A1 was obtained.

After the liquid A1 obtained was cooled to 90° C., a solution in which 140 parts of cesium bicarbonate was dissolved in 250 parts of pure water was added under stirring with a rotor stirrer, and thus a liquid A was prepared. The pH of the liquid A was 0.8.

The liquid A obtained was stirred with a rotor stirrer for 15 minutes. Next, a solution in which 130 parts of ammonium bicarbonate was dissolved in 700 parts of pure water was added to the liquid A, and further stirred for 20 minutes, and thus a liquid B was obtained. The value of v/M here is shown in Table 1. The pH of the liquid B obtained was 4.2.

An aqueous 65% nitric acid solution was added to the liquid B obtained, to adjust the pH to 1.5, and then stirred for 20 minutes, and thus a liquid C was obtained.

The liquid C obtained was dried with a co-current system spray dryer under conditions of a dryer inlet temperature of 300° C. and a rate of rotation of a disc for slurry spraying of 18,000 rpm, and thus a catalyst powder having an average particle size of 25 μm was obtained. The catalyst composition excluding oxygen, of the catalyst powder, was P_{1.4}Mo₁₂V_{0.8}Cu_{0.05}Cs_{1.25} (NH₄)_{3.6}. The measurement result of the penetration rate coefficient of the catalyst powder to ethyl alcohol is shown in Table 1.

Four parts of hydroxypropyl methylcellulose and 22 parts of ethyl alcohol based on 100 parts of the catalyst powder obtained were kneaded and thus mixed by a batch type kneading machine equipped with a double arm sigma blade, until a clayey product was obtained, and thus a catalyst powder mixture was obtained.

The catalyst powder mixture obtained was extrusion-molded with a plunger-type extruder, and molded into a columnar shape having an outer diameter of 5.5 mm and a length of 5.5 mm. Next, such a columnar product was dried with a hot-air dryer at 90° C. for 8 hours, and thus a catalyst molded article was obtained. The measurement result of the falling pulverization ratio of the catalyst molded article is shown in Table 1.

A reaction tube was filled with the catalyst molded article obtained, so that the filling volume in the reaction tube was 2500 mL, and was fired under flow of air at 370° C. for 17 hours. Next, oxidation reaction of methacrolein was performed at 290° C. with a raw material gas of 6% by volume of methacrolein, 12% by volume of oxygen, 10% by volume of water vapor, and 72% by volume of nitrogen for a contact time of 2.9 seconds. The results are shown in Table 1.

### Example 2

A liquid B was obtained by the same method as in Example 1.

An aqueous 65% nitric acid solution was added to the liquid B obtained, to adjust the pH to 2.5, and then stirred for 20 minutes, and thus a liquid C was obtained.

The liquid C obtained was dried by the same method as in Example 1, and thus a catalyst powder having an average particle size of 25 μm was obtained. The catalyst composition excluding oxygen, of the catalyst powder, was P_{1.4}Mo₁₂V_{0.8}Cu_{0.05}Cs_{1.25} (NH₄)_{3.6}. The measurement result of the penetration rate coefficient of the catalyst powder to ethyl alcohol is shown in Table 1.

The catalyst powder obtained was used to obtain a catalyst powder mixture by the same method as in Example 1.

The catalyst powder mixture obtained was used to perform molding by the same method as in Example 1, and thus a catalyst molded article was obtained. The measurement result of the falling pulverization ratio of the catalyst molded article is shown in Table 1.

The catalyst molded article obtained was used to perform firing and oxidation reaction of methacrolein by the same method as in Example 1. The result is shown in Table 1.

### Example 3

A liquid B was obtained by the same method as in Example 1.

An aqueous 65% nitric acid solution was added to the liquid B obtained, to adjust the pH to 3.7, and then stirred for 20 minutes, and thus a liquid C was obtained.

The liquid C obtained was dried by the same method as in Example 1, and thus a catalyst powder having an average particle size of 25 μm was obtained. The catalyst composition excluding oxygen, of the catalyst powder, was P_{1.4}Mo₁₂V_{0.8}Cu_{0.05}Cs_{1.25} (NH₄)_{3.6}. The measurement result of the penetration rate coefficient of the catalyst powder to ethyl alcohol is shown in Table 1.

The catalyst powder obtained was used to obtain a catalyst powder mixture by the same method as in Example 1.

The catalyst powder mixture obtained was used to perform molding by the same method as in Example 1, and thus a catalyst molded article was obtained. The measurement result of the falling pulverization ratio of the catalyst molded article is shown in Table 1.

The catalyst molded article obtained was used to perform firing and oxidation reaction of methacrolein by the same method as in Example 1. The result is shown in Table 1.

### Example 4

A liquid B was obtained by the same method as in Example 1 except that the value of v/M was modified as shown in Table 1.

An aqueous 65% nitric acid solution was added to the liquid B obtained, to adjust the pH to 1.5, and then stirred for 20 minutes, and thus a liquid C was obtained.

The liquid C obtained was dried by the same method as in Example 1, and thus a catalyst powder having an average particle size of 25 μm was obtained. The catalyst composition excluding oxygen, of the catalyst powder, was P_{1.4}Mo₁₂V_{0.8}Cu_{0.05}C_{1.25}(NH₄)_{3.6}. The measurement result of the penetration rate coefficient of the catalyst powder to ethyl alcohol is shown in Table 1.

The catalyst powder obtained was used to obtain a catalyst powder mixture by the same method as in Example 1.

The catalyst powder mixture obtained was used to perform molding by the same method as in Example 1, and thus a catalyst molded article was obtained. The measurement result of the falling pulverization ratio of the catalyst molded article is shown in Table 1.

The catalyst molded article obtained was used to perform firing and oxidation reaction of methacrolein by the same method as in Example 1. The result is shown in Table 1.

### Example 5

A catalyst powder was obtained by the same method as in Example 1. The measurement result of the penetration rate coefficient of the catalyst powder to isopropyl alcohol is shown in Table 1.

A catalyst powder mixture was obtained by the same method as in Example 1, except that the catalyst powder obtained was used, and isopropyl alcohol was used instead of ethyl alcohol.

The catalyst powder mixture obtained was used to perform molding by the same method as in Example 1, and thus a catalyst molded article was obtained. The measurement result of the falling pulverization ratio of the catalyst molded article is shown in Table 1.

The catalyst molded article obtained was used to perform firing and oxidation reaction of methacrolein by the same method as in Example 1. The result is shown in Table 1.

### Comparative Example 1

A liquid B was obtained by the same method as in Example 1.

The liquid B obtained was dried by the same method as in Example 1, and thus a catalyst powder having an average particle size of 25 μm was obtained. In other words, a catalyst powder was obtained without step (2) carried out. The catalyst composition excluding oxygen, of the catalyst powder, was P_{1.4}Mo₁₂V_{0.8}Cu_{0.05}Cs_{1.25}(NH₄)_{3.6}. The measurement result of the penetration rate coefficient of the catalyst powder to ethyl alcohol is shown in Table 1.

The catalyst powder obtained was used to obtain a catalyst powder mixture by the same method as in Example 1.

The catalyst powder mixture obtained was used to perform molding by the same method as in Example 1, and thus a catalyst molded article was obtained. The measurement result of the falling pulverization ratio of the catalyst molded article is shown in Table 1.

The catalyst molded article obtained was used to perform firing and oxidation reaction of methacrolein by the same method as in Example 1. The result is shown in Table 1.

### Comparative Example 2

A liquid A was obtained by the same method as in Example 1.

The liquid A obtained was stirred with a rotor stirrer for 15 minutes. Next, a solution in which 130 parts of ammonium bicarbonate was dissolved in 700 parts of pure water was added to the liquid A, and further stirred for 20 minutes. Next, an aqueous 65% nitric acid solution was added to adjust the pH to 1.5 and then stirred for 20 minutes, and thus a liquid B was obtained. The value of v/M here is shown in Table 1.

The liquid B obtained was dried by the same method as in Example 1, and thus a catalyst powder having an average particle size of 25 μm was obtained. In other words, a catalyst powder was obtained without step (2) carried out. The catalyst composition excluding oxygen, of the catalyst powder, was P_{1.4}Mo₁₂V_{0.8}Cu_{0.05}Cs_{1.25}(NH₄)_{3.6}. The measurement result of the penetration rate coefficient of the catalyst powder to ethyl alcohol is shown in Table 1.

The catalyst powder obtained was used to obtain a catalyst powder mixture by the same method as in Example 1.

The catalyst powder mixture obtained was used to perform molding by the same method as in Example 1, and thus a catalyst molded article was obtained. The measurement result of the falling pulverization ratio of the catalyst molded article is shown in Table 1.

The catalyst molded article obtained was used to perform firing and oxidation reaction of methacrolein by the same method as in Example 1. The result is shown in Table 1.

### Comparative Example 3

A liquid B was obtained by the same method as in Example 1 except that the value of v/M was modified as shown in Table 1.

An aqueous 65% nitric acid solution was added to the liquid B obtained, to adjust the pH to 1.5, and then stirred for 20 minutes, and thus a liquid C was obtained.

The liquid C obtained was dried by the same method as in Example 1, and thus a catalyst powder having an average particle size of 25 μm was obtained. The catalyst composition excluding oxygen, of the catalyst powder, was, P_{1.4}Mo₁₂V_{0.8}Cu_{0.05}Cs_{1.25}(NH₄)_{3.6}. The measurement result of the penetration rate coefficient of the catalyst powder to ethyl alcohol is shown in Table 1.

The catalyst powder obtained was used to obtain a catalyst powder mixture by the same method as in Example 1.

The catalyst powder mixture obtained was used to perform molding by the same method as in Example 1, and thus a catalyst molded article was obtained. The measurement result of the falling pulverization ratio of the catalyst molded article is shown in Table 1.

The catalyst molded article obtained was used to perform firing and oxidation reaction of methacrolein by the same method as in Example 1. The result is shown in Table 1.

TABLE 1

| | pH | | | | | | | | |
|---|---|---|---|---|---|---|---|---|---|
| | | | | Difference | | | Penetration | Falling | Yield of |
| | | | | in pH | v/M | | rate | pulverization | methacrylic |
| | Liquid | Liquid | Liquid | Liquid B − | [mol/ | Type of | coefficient | ratio | acid |
| | A | B | C | Liquid C | (hr · kg)] | liquid | [g²/s] | [%] | [%] |
| Example1 | 0.8 | 4.2 | 1.5 | 2.7 | 0.43 | Ethyl | 0.0938 | 0.2 | 70.8 |
| | | | | | | alcohol | | | |
| Example2 | 0.8 | 4.2 | 2.5 | 1.7 | 0.43 | Ethyl | 0.0774 | 0.4 | 70.5 |
| | | | | | | alcohol | | | |
| Example3 | 0.8 | 4.2 | 3.7 | 0.5 | 0.43 | Ethyl | 0.0742 | 0.4 | 70.3 |
| | | | | | | alcohol | | | |
| Example4 | 0.8 | 4.4 | 1.5 | 2.9 | 0.96 | Ethyl | 0.0904 | 0.2 | 70.8 |
| | | | | | | alcohol | | | |
| Example5 | 0.8 | 4.2 | 1.5 | 2.7 | 0.43 | Isopropyl | 0.0745 | 0.3 | 70.4 |
| | | | | | | alcohol | | | |
| Comparative | 0.8 | 4.2 | — | — | 0.43 | Ethyl | 0.0697 | 2.5 | 68.8 |
| Example1 | | | | | | alcohol | | | |
| Comparative | 0.8 | 1.5 | — | — | 0.43 | Ethyl | 0.0689 | 2.3 | 68.8 |
| Example2 | | | | | | alcohol | | | |
| Comparative | 0.8 | 4.8 | 1.5 | 3.3 | 3.83 | Ethyl | 0.0665 | 2.6 | 68.5 |
| Example3 | | | | | | alcohol | | | |

As shown in Table 1, Examples each using the method allowing the penetration rate coefficient of the liquid to the catalyst powder to be within the prescribed range could provide a catalyst molded article high in mechanical strength and enabling methacrylic acid to be produced at a high yield. It is expected that the catalyst molded article can be used to perform long-term continuous running, thereby suppressing an increase in differential pressure in a reactor and allowing a high yield of methacrylic acid to be kept over a long period.

Here, methacrylic acid obtained in the present Examples can be esterified, thereby obtaining methacrylic acid ester.

What is claimed is:

1. A method for producing a catalyst molded article for use in the production of unsaturated carboxylic acid by oxidation reaction of unsaturated aldehyde, the method comprising
(1) a step of adding an ammonium radical to a solution or slurry (liquid A) including molybdenum and phosphorus, at a rate satisfying the following expression (I), to prepare a solution or slurry (liquid B),
$$0.1 \leq v / M \leq 3 \qquad ( I )$$
wherein v represents the rate of addition of the ammonium radical [mol/hour], and M represents the mass of the liquid A [kg],
(2) a step of adjusting the pH of the liquid B, to prepare a solution or slurry (liquid C) having a pH lower than that of the liquid B by 0.2 or more and having a pH of 4 or less,
(3) a step of drying the liquid C, to obtain a catalyst powder,
(4) a step of mixing the catalyst powder and a liquid, to produce a catalyst powder mixture, and
(5) a step of molding the catalyst powder mixture, to produce a catalyst molded article, wherein
the penetration rate coefficient of the liquid to the catalyst powder in step (4) is 0.07 g²/s or more.

2. The method for producing a catalyst molded article according to claim 1, wherein the penetration rate coefficient of the liquid to the catalyst powder in step (4) is 0.073 to 0.15 g²/s.

3. The method for producing a catalyst molded article according to claim 1, wherein the liquid contains one selected from the group consisting of water and an alcohol having 1 to 4 carbon atoms, in step (4).

4. The method for producing a catalyst molded article according to claim 1, wherein the liquid contains 50% by mass or more of one selected from the group consisting of water and an alcohol having 1 to 4 carbon atoms, in step (4).

5. The method for producing a catalyst molded article according to claim 1, wherein the catalyst powder mixture is extrusion-molded to produce the catalyst molded article in step (5).

6. The method for producing a catalyst molded article according to claim 1, wherein the pH of the liquid A is 3 or less in step (1).

7. The method for producing a catalyst molded article according to claim 1, wherein the v/M is 0.2 to 2 in step (1).

8. The method for producing a catalyst molded article according to claim 1, wherein the liquid B is stirred for 5 minutes or more and then the pH is adjusted in step (2).

9. The method for producing a catalyst molded article according to claim 1, wherein the liquid C having a pH lower than that of the liquid B by 0.5 or more is prepared in step (2).

10. The method for producing a catalyst molded article according to claim 1, wherein the liquid C having a pH of 3.5 or less is prepared in step (2).

11. The method for producing a catalyst molded article according to claim 1, wherein the catalyst powder has a composition represented by the following formula (II):
PₐMo_{b}V_{c}Cu_{d}X1ₑY1_{f}Z1_{g}(NH₄)ₕOᵢ (II)
wherein P, Mo, V, Cu, NH₄ and O respectively represent phosphorus, molybdenum, vanadium, copper, ammonium radical and oxygen, X1 represents at least one element selected from the group consisting of silicon, germanium, arsenicum, antimony, and bismuth, Y1 represents at least one element selected from the group consisting of niobium, tungsten, iron, zinc, chromium, cobalt and manganese, Z1 represents at least one element selected from the group consisting of lithium, sodium, potassium, rubidium and cesium, a to i each represent the molar ratio of each component, b=12, a=0.5 to 3, c=0.01 to 3, d=0.01 to 2, e=0 to 3, f=0 to 3, g=0.01 to 3, and h=0.01 to 30 are satisfied, and i is the molar ratio of oxygen necessary for satisfying the valence of such each component.

12. A method for producing unsaturated carboxylic acid, comprising producing unsaturated carboxylic acid with a catalyst molded article produced by the method according to claim 1, by oxidation reaction of unsaturated aldehyde.

13. A method for producing unsaturated carboxylic acid ester, comprising esterifying unsaturated carboxylic acid produced by the method according to claim 12.
